(19) United States
(12) Patent Application Publication
(10) Pub. No.: US 2012/0238630 A1
(43) Pub. Date: Sep. 20, 2012

(54) COMPOSITIONS COMPRISING NONSTEROIDAL ANTI - INFLAMMATORY DRUGS

(75) Inventors: Giuseppe Claudio Viscomi, Bologna (IT); Giuseppe Bottoni, Bologna (IT); Paola Maffei, Bologna (IT); Annalisa Sforzini, Bologna (IT); Jackie Leeder, Bologna (IT); Vittoria Lauro, Bologna (IT)

(73) Assignee: Alfa Wassermann S.P.A., Alanno (Pescara) (IT)

(21) Appl. No.: 13/512,848

(22) PCT Filed: Nov. 17, 2010
(86) PCT No.: PCT/IB2010/055228

§ 371 Date: May 30, 2012

(30) Foreign Application Priority Data
Dec. 1, 2009 (IT) BO2009A000775

(51) Int. Cl. A61K 47/36; A61P 1/04; A61P 25/22; A61K 31/192; A61P 37/08; A61P 11/14; A61P 11/12; A61P 29/00; A61P 1/08

(52) U.S. Cl. 514/570; 514/777

(57) ABSTRACT

The present invention describes liquid compositions comprising pharmaceutically active principles, like scarcely water-soluble, nonsteroidal anti-inflammatory drugs, in particular, but not exclusively, flurbiprofen (FP), processes for their manufacture and their therapeutic use. The liquid compositions comprising FP in association with natural polymers, like xyloglucans (XG), and pharmaceutically acceptable excipients like glycerol, are suitable to be used for pharmaceutical uses and are characterized by good tolerability, therapeutic efficacy, stability and palatability.

### OBJECT OF THE INVENTION

The present invention describes liquid compositions comprising pharmaceutically active principles, like scarcely water-soluble, nonsteroidal anti-inflammatory drugs, in particular, but not exclusively, flurbiprofen (FP), processes for their manufacture and their therapeutic use.

The liquid compositions comprising FP in association with natural polymers like xyloglucans (XG) and pharmaceutically acceptable excipients, are suitable to be used for pharmaceutical uses and are characterized by good tolerability, therapeutic efficacy, stability and palatability.

### BACKGROUND OF THE INVENTION

The present invention can be applied to acid derivatives of nonsteroidal anti-inflammatory drugs, such as, for instance, ibuprofen, naproxen, fenoprofen, ketoprofen, indoprofen, carprofen, miroprofen, tiaxoprofen, alminoprofen, tiaprofenic acid and flurbiprofen.

The main mechanism of action of nonsteroidal anti-inflammatory drugs FANS is the inhibition of the cyclooxygenase activity (COX), with a following reduction of the prostaglandin synthesis.

COX converts arachidonic into prostaglandin H2 (PGH2), which is then metabolized in different prostanoids according to the kind of cells or tissue.

Two COX isoforms have been identified: COX1, constitutive and expressed in several tissues, and COX2, generally induced by pro-inflammatory stimuli.

With a reference to the present invention, the favorite elements in the group of active principles belonging to derivates of propionic acid comprise flurbiprofen, naproxen, ketoprofen, ibuprofen and, more in particular, flurbiprofen.

Flurbiprofen, 2-(2-fluoro-4-phenyl-phenyl) propionic acid, hereinafter FP, is well known for its anti-inflammatory, antipyretic and analgesic activity, for the treatment of pain, of inflammatory and feverish condition, e.g. in arthritis, osteoarthritis, ankylosing spondytilis, post surgical pain, post partum pain and cutaneous injuries.

FP molecule exists in two enantiomeric forms, and the term FP means here both the mixture of these two enantiomers in any possible proportion, here indicated as racemic formula, and the two single enantiomers.

FP can exist in the form of pharmaceutically acceptable salts or in the form of ester derivatives, more soluble in water than the acid form.

Pharmaceutical compositions containing FP are known and commercially available in different forms, such as, for instance, tablets, lozenges, collutories, plasters and nebulizer solutions. In particular, the formulations in lozenges, collutories and nebulizer solutions are useful in the treatment of inflammations of the oropharyngeal cavity, releasing useful therapeutic amounts of FP in the oral cavity.

Paganelli et al. describes in Minerva Stomatol. 1993, 42(6), 271-274 that FP, administered by oromucosal route, is safe and well-tolerated; the advantage of oral administrations by means of oromucosal nebulizers is represented by the fact that suitable amounts of active principle are directly and exclusively released in the interested area, thus creating a better balance between therapeutic efficacy and induction of adverse reactions due to a scarce systemic absorption.

EP 0 862 424 describes pharmaceutical compositions containing FP and mono- or disaccharide sugars in the form of tablets or lozenges, useful against inflammations involving the oropharyngeal cavity, wherein the active principle is directly released in the interested area.

FP, like any other nonsteroidal anti-inflammatory agent, when administered in the oropharyngeal cavity, gives an unpleasant burning feeling badly tolerated by patients, above all when they must be treated for long periods, as described by Breslin P. A. et al. in Chem. Sens. 26, 55-65, 2001.

Many solid formulations have been studied, with techniques and ingredients available to the person skilled in the art for finding formulations able to improve the acceptability to patients.

U.S. Pat. No. 6,194,003 describes solid formulations containing FP with the addition of sweeteners and salts, which reduce the irritating stimulus in the oral cavity and improve palatability.

Because of the difficult solubilization of FANS active principles in their acid form in water, the development of solid formulations has been preferred to liquid formulations, as in the above reported examples.

Liquid formulations are reported, for instance, in WO 2005/058276, describing liquid formulations containing FP obtained by solubilization in the presence of ethanol 95° with the addition of polysorbate surfactants in order to prevent the creation of precipitates during their conservation.

WO 2005/058276 tries to solve the problem of taste acceptability with the addition of tris(hydroxymethyl)aminomethane and with sugars such as sorbitol, saccharine, maltilol, sucrose.

Sucrose, like other mono- and disaccharide sugars, is badly accepted in formulations, since patients affected by diabetes cannot assume it for the deficiency of insulin.

The use of polysorbates, however, is not an optimal solution for pharmaceutical formulations, since some adverse reactions can possibly arise from their use. In fact, cases of hypersensitivity and local inflammation have been reported, and in pediatrics, cases of thrombocythemia, hepatic and kidney dysfunctions, ascites, local metabolic acidosis have been reported, and cases of decease associated to hepatic and pulmonary diseases in children with a low body weight have been described (Martindale 32 Ed. 1999, page 1328).

U.S. Pat. No. 5,183,829 describes liquid formulations containing FP in its acid form, in the presence of glycols, ethanol, polyols and disperging agents in order to prevent the formation of agglomerates in gastric juices with a following burning and irritating sensation.

Pharmaceutical compositions in solutions containing alcohols for oral use cause dryness and irritation of the oropharyngeal mucosae and are therefore scarcely tolerated for longer periods of treatment, in particular in pediatric formulations.

EP 0 253 472 tries to solve the problem of FB solubilization by emulsifying the active principle in soy oil in the presence of phospholipids.

Compositions containing FP for oromucosal applications, such as mouth rinsing/washing solutions, oral nebulizations and tablets, play a very relevant role in the therapy of oropharyngeal diseases such as gingivitis, stomatitis, pharyngitis, pains after dental surgery or periodontal pains.

The use of organic solvents, in particular of ethyl alcohol, has huge drawbacks because of their inflammability, in particular when they are used in large amounts.

Therefore, there is a need to provide efficacious formulations containing FP, with an improved palatability and acceptability to patients, free from alcohols and surfactants, such as, e.g., nebulizer solutions, collutories or aerosol solutions. These formulations would represent a valid alternative to solid formulations, like tablets, since, thanks to local administration and action, they show a higher efficacy at lower doses, with the clear advantage of reducing systemic adverse events, such as ulcers and bleedings of the gastro-intestinal duct. Moreover, there is the further need to have formulations which are not irritating for the mucosa of the oral cavity and, at the same time, have a pleasant and acceptable taste, namely a good palatability.

It has been surprisingly found, and this is the object of the present invention, that when the nonsteroidal anti-inflammatory drugs, and in particular FP, are added to natural polymers, like xyloglucans, the obtained aqueous formulations are stable at room temperature, do not need organic solvents or surfactants for their solubilization and have a good palatability, even without added sweeteners. The xyloglucans added to the solution of nonsteroidal anti-inflammatory drugs, and in particular to FP, are a class of polysaccharides structurally related to cellulose and strictly associated to it in the cell wall of superior plants. Furthermore, they are one of the main components, probably with a function of energy reservoir, of seeds of plants such as Tamarindus Indica, originally coming from India and South-East Asia, Detarium Senegalense Gmeli, diffused in Africa (in particular in Nigeria), Afzelia Africana, diffused both in Central and East Africa and in the coast savannas and forests of East Africa, and Jatoba.

Xyloglucans are characterized by a main chain of (1,4)-β-D-Glucan substituted with side chains of α-D-xylopyranose and β-D-galactopyranosil-(1,2)-α-D-xylopyranose bound by means of α(1,6) bond to glucan residues. The distribution of the residues in the side chains is different in xyloglucans of different species. In the xyloglucan structure three oligomeric units have been identified, namely nonasaccharides, octasaccharides and heptasaccharides, which are distinguished by the number of carbohydrates in the side chains bound to the galactose molecules of the main chain, as reported by U. Hiroshi in Trends in Glycoscience and Glycotechnology, 14, 355-376, 2002.

The main application of xyloglucans is human and animal feeding. Moreover, the flour obtained from the seeds of these plants, containing xyloglucans, is commonly used in the food industry as stabilizing, jellifying and thickening agent. This makes the product acceptable for oral administration.

Sporadically, the use of xyloglucans has also been reported in medical devices or as a component in pharmaceutical preparations, as described in EP 0892636.

The present invention describes liquid formulations containing FP, releasing the active principle with a proven therapeutic efficacy, free from safety contraindications and with a good acceptability to patients.

If compared to what described in the prior art literature, the found formulation not only solves the problem of palatability without employing the usual sugar addition, but even confers a higher therapeutic efficacy providing mucoadhesive solutions which, thanks to their characteristics, increase the contact time with the anatomic oropharyngeal portion subjected to an inflammatory process.

A further aspect of the present invention is the obtainment of clear aqueous solutions containing nonsteroidal anti-inflammatory drugs in their acid form.

A further aspect of the present invention is represented by the fact that the liquid compositions containing the nonsteroidal anti-inflammatory drug can be used as such or in the presence of pharmaceutically acceptable excipients for oral formulations, in the form of nebulizer solutions, collutories, emulsions or aerosol solutions, or for preparations of other pharmaceutical forms, such as creams, ointments, granulates, tablets or plasters without limitations.

A further aspect of the present invention consists in that the nonsteroidal anti-inflammatory drug contained in the liquid compositions can be associated to other anti-inflammatory drugs or other pharmaceutical active principles.

### DESCRIPTION OF THE INVENTION

The present invention describes liquid compositions comprising at least a nonsteroidal anti-inflammatory drug, in particular FP, for the preparation of collutories, nebulizer solutions, emulsions, syrups, stable at room temperature and in particular with a palatable taste, free of burning or irritating sensation even without adding sugars or sweeteners.

In particular, the present invention describes aqueous pharmaceutical compositions comprising the nonsteroidal anti-inflammatory drug, and in particular FP, associated to a natural polymer having a xyloglucan structure and glycerol. The used xyloglucan is obtained from vegetable extracts of xyloglucans, purified as described in EP 1 898 876 B1 and characterized by a viscosity comprised between 150 and 800 mPa·sec at a temperature of 25° C. operating with a shear rate of 200 seconds⁻¹ and a rest time of 15 minutes and an absorbance value at 280 nm lower than 0.5 absorbance units (abs) in a 2% by weight aqueous solution at a temperature of 25° C.

The aqueous liquid compositions comprise FP at a concentration comprised between 0.05% and 10.0% by weight; xyloglucan at a concentration comprised between 0.05% and 5.0% by weight and glycerol at a concentration comprised between 5.0% and 70.0% by weight. The concentrations are such that the administration of 200 μl aliquots, for instance with nebulizers, at a concentration comprised between 0.05% and 10.0%, gives absolute amounts of FP comprised between 100 μg and 20.0 mg.

The compositions are stable, are able to release FP in the oral cavity, have a good palatability with no burning sensation and do not show contraindications, so that they can be administered to children, since they are free from organic solvents and in particular free from alcohols. Moreover, the described compositions can be administered also to patients affected by diabetes, since xyloglucan is not digested by gastrointestinal enzymes.

Pharmaceutical compositions characterized by the presence of glycerol and xyloglucan show emollient and mucoadhesive properties, with the advantage of a longer time of contact of the active principle with the inflamed anatomic part.

This involves a higher therapeutic efficacy even at low doses, a more lasting effect after each single administration with a following reduction of the number of administrations and with a lower toxicity if compared to aqueous or hydro-alcoholic solutions.

The pharmaceutical compositions described in the examples, and non limiting of the invention, have been evaluated with regard to their efficacy, safety, tolerability and acceptability.

The pharmaceutical compositions object of the present invention have been prepared by adding FP in a water volume containing salts like phosphates and/or carbonates, in an amount comprised between 0.05% and 10.0%, preferably between 0.1% and 5.0% by weight if compared to the final solution.

Then, xyloglucan has been added to the aqueous solution in a range of concentrations comprised between 0.05% and 10.0% by weight, preferably between 0.1% and 5.0% by weight if compared to the weight of the final solution, and the solution is kept under stirring at room temperature till complete dissolution.

Glycerol has been added to the homogeneous solution in a range of concentrations comprised between 5.0% and 70.0%, preferably between 10.0% and 30.0% by weight, and the resulting solution is kept under stirring at room temperature for a time varying between 10 minutes and 1 hour according to the amount for obtaining a homogeneous solution.

The pharmaceutical compositions can contain stabilizers, aromatizing agents, edulcorating agents, antiseptic or antibacterial agents such as benzoic acid or sodium benzoate. One or more sugars such as sorbitol, xylitol, maltitol, lactitol, mannitol or sodium saccharin, saccharose, dextrose and/or excipients and/or fruit or natural herbs fragrances and/or colorants can be added to the resulting solution.

Example 1 describes a liquid composition useful for oral preparations such as nebulizer solutions, collutories, emulsions, aerosol solutions, syrups or for being added to excipients for different pharmaceutical formulations, obtained by adding 250 mg of FP to a saline solution constituted by 440 mg of sodium phosphate monobasic monohydrate, 250 mg of sodium carbonate monohydrate and 100 mg of ethylenediaminetetraacetic acid (EDTA). The solution is kept under stirring at room temperature and, always under stirring, 400 mg of xyloglucan have been added. The solution is kept under stirring for ca. 2 hours at room temperature till complete solubilization, then 30 g of glycerol are added. The aqueous solution is then brought to basic pH in a range comprised between pH 8.0 and pH 8.5 with the addition of sodium hydroxide, filtered and put in containers for its storage.

The relative percentages of the components in the solution are reported in Table 1.

Example 2 describes a liquid comparison composition containing FP wherein, as an alternative to XG, ethanol and a surfactant are added to solubilize the active principle. Example 2 is given for demonstrating that, in the absence of the components of the formulation object of the present invention, it is possible to obtain an equivalent aqueous solution containing FP with the simple addition of ethanol at a final concentration of ca. 10% and of hydrogenated castor oil belonging to the class of polysorbates.

The solution is prepared by adding FP to a solution constituted by ethanol 96°, methyl parahydroxybenzoate, propyl parahydroxybenzoate, polyoxyethylenated hydrogenated castor oil 40, and the obtained solution is kept under stirring till complete solubilization.

The clear alcohol solution is then added to an aqueous solution containing sugars, saccharine and citric acid, and the pH is brought to a value comprised between pH 6.5 and pH 7.5. The solution is filtered and placed in containers for its storage. The percentages of the various components are reported in Table 2.

Compositions comprising FP and xyloglucans, possibly with the addition of glycerol, in the absence of sugars can be used for preparing collutories useful against ocular inflammations, or vaginal lavages, wherein the anti-inflammatory action is associated to the beneficial action of xyloglucan, conferring humidity to the mucosa.

The preparations comprising FP object of the present invention are stable at room temperature and can be stored in storage containers or distributed in suitable containers, possibly provided with dosing device and nebulizer pump.

The liquid compositions comprising FP, xyloglucan and glycerol can also comprise other water soluble or insoluble active principles for a desired local effect or for the mucosa absorption. The active principles in association can have different activity or can have different therapeutic indications, and their association can lead to a higher therapeutic efficacy.

The liquid compositions containing FP, object of the present invention, have been tested in a clinical study to evaluate the local and systemic tolerability after repeated dosages and to determine the kinetic profiles indicating the levels of systemic absorption. The solutions have been administered to 26 healthy volunteers, among whom 16 were males and 10 females, aged between 18 and 55.

The healthy volunteers received the pharmaceutical composition prepared according to Example 1 in two 200 μl nebulizations three times a day, for a seven-day period, then a single administration on the eighth day.

The local tolerability of the formulation has been determined by evaluating, in different days of the treatment, the possible presence of erythema, edema, hemorrhagic petechiae and ulcers in the oral cavity.

Furthermore, it has been asked to the volunteers to remark a possible onset, in the mouth, of itch, pain or burning sensation. With regard to the palatability it has been further asked to the healthy volunteers to express their opinion at the time of the nebulization in the oral cavity and 30 minutes after the administration.

No one showed edema, hemorrhagic petechiae or mouth ulcers; only one subject showed a light erythema on the fourth day of treatment, which disappeared after two days with no further treatment.

In the subjective evaluation three cases of light itch and five cases of light burning sensation were reported by four volunteers, after 572 total administrations.

No one complained about a possible scarce acceptability of the composition in question with regard to its palatability.

With regard to the safety of the preparation, the complete objective examination, the laboratory chemical-clinical analysis and the electrocardiogram did not show any difference between the periods before and after the treatment.

Five subjects showed adverse events of scarce/moderate severity, which disappeared with no further treatment and which were interpreted as non related to the drug.

The composition comprising FP prepared according to Example 1 turned out to be optimally tolerated both from a local and from a systemic point of view.

The tolerability of the liquid formulation must be intended as tolerability to FP in the formulation, to xyloglucan and to their combination.

In particular, the tolerability to xyloglucan in the formulation containing FP must be intended as extended to all nonsteroidal acid anti-inflammatory drugs and to their combination.

This tolerability must also be intended as extended to the same solution administered to the liquid composition in all administrations.

The pharmacokinetics trial has been carried out to confirm that the developed formulation is able to release the active principle directly on the anatomic site interested by the inflammatory process (oropharyngeal cavity), where it is absorbed in an amount about 10 times lower than solutions having the same concentration administered by oral route as described by Stalker D. J. et al. in Pharm. Res. 1991; 8(5):605-607 and Gonzalez-Younes I et al. in J. Pharm. Sci. 1991; 80:820-823.

During the period of administration of the composition object of the present invention, blood samples were taken before each administration and on days 1, 4, 6, 7 and 8.

The values of the obtained plasma concentrations demonstrated that there is no FP accumulation after repeated administrations.

After the end of the treatment, the FP concentration has been determined for further 24 hours corresponding to hour 1, 2, 3, 4, 6, 8, 10, 12, 16 and 24 after the last administration.

The obtained values are reported in Example 3 and summarized in Table 4.

The active principle was rapidly adsorbed by the oral mucosa, thus reaching a maximum plasma concentration Cₘₐₓ corresponding to 0.182±0.050 μg/ml 1 hour after the last administration, and the removal time (T_{1/2}) turned out to correspond to 4.47±1.7 hours. The area subtended to the AUC₀₋₂₄ curve turned out to correspond to 0.94 μg/ml·h and AUC_{0-inf.} turned out to correspond to 1.14 μg/ml·h, ca. 20% higher than the AUC at the last point.

The FP systemic concentrations turned out to be very low, demonstrating a low risk of adverse events, usually provoked by FANS, for example gastrointestinal ones, but with systemic activities.

The compositions of the present invention can therefore be used for derivates of propionic acid comprise flurbiprofen, naproxen, ketoprofen, ibuprofen and flurbiprofen at concentrations higher than those reported in the examples, with a higher total number of daily administrations, thanks to the fact that it has been demonstrated that the active principle does not accumulate in blood and does not show toxic effects.

Because of the similarity of chemical structure and action mechanisms, the described formulation containing a nonsteroidal anti-inflammatory drug can be extended to all nonsteroidal acid anti-inflammatory drugs and to their derivatives, without limitation.

The described compositions can include gastroprotectors, antiemetics, antiulcer drugs, anti-stress or ansiolitic agents, antiacids, inhibitors of gastric secretions such as anticholinergics, inhibitors of muscarinic receptor, antibacterials, antitussives, mucolitics, antistaminics.

Moreover, the compositions can contain vitamins, amino acids, other natural extracts or mineral compounds.

When other active principles are associated to the nonsteroidal anti-inflammatory drug, and in particular to FP, the dosage is not limited to a specific component, but to the mixture of several FANS.

Furthermore, FANS can be prepared and administered in the same composition or prepared and administered separately.

The proportions of other active principles can be, for instance, comprised between 0.01 and 500 parts by weight.

The described examples demonstrating the invention must not be intended as limiting of the invention.

### Example 1

### Preparation of a Liquid Composition Containing FP

Sodium phosphate monobasic monohydrate, sodium carbonate monohydrate, EDTA and sodium benzoate in the amounts reported in Table 1 are added to ca. 50 ml of water, and the solution is kept under stirring for ca. 10 minutes at room temperature. 250 mg of FP are added, and the solution is kept under stirring for ca. 1 hour, then xyloglucan is added. The resulting solution is kept under stirring for ca. 2 hours, turning clear.

Sodium saccharin in a quantity corresponding to 0.15 g and glycerol are added to the solution, and it is kept under stirring for 1 hour.

The solution of sodium hydroxide is added dropwise to obtain a pH value in a range comprised between pH 8.0 and pH 8.5, and then distilled water is added till reaching a volume of 100 ml.

The solution is filtered under vacuum using polypropylene filters having a 6 μm porosity.

The solution is placed in glass containers or in containers provided with a pump or adapter for being used as nebulizer solution, colluttory or aerosol solution.

TABLE 1

| | Amount | Concentration % |
|---|---|---|
| Compound | (grams) | (w/w) |
| Flurbiprofen | 0.25 | 0.25% |
| Glycerol | 30.00 | 30.0% |
| Xyloglucan | 0.40 | 0.4% |
| Sodium phosphate monobasic | 0.44 | 0.44% |
| monohydrate (NaH₂PO₄•H₂O) | | |
| Sodium carbonate monohydrate | 0.25 | 0.25% |
| (Na₂CO₃•H₂O) | | |
| EDTA | 0.10 | 0.10% |
| Sodium benzoate | 0.25 | 0.25% |
| Balsamic peppermint fragrance | 0.20 | 0.20% |
| Sodium saccharin | 0.15 | 0.15% |
| Sodium hydroxide | a.r. at pH 8.0-8.5 | |
| Distilled water | a.r. to 100 ml | |

### Example 2

### Preparation of a Liquid Composition Containing FP, Ethanol and Surfactant

Sorbitol, glycerol and sodium saccharin are added to ca. 50 ml of water in the amounts reported in Table 2, and the solution is kept under stirring till complete dissolution. Then citric acid and the patent blue V dye have been added.

In another container ethanol 96°, methyl parahydroxybenzoate and propyl parahydroxybenzoate, polyoxyethylenated hydrogenated castor oil 40 are added and kept under stirring till complete dissolution. Finally, FP and peppermint fragrance are added.

The alcohol phase is then transferred into the container containing the aqueous solution and the pH is in case corrected by adding sodium hydroxide, in a pH range comprised between pH 6.5 and pH 7.5.

The solution is then brought to a volume of 100 ml with water, and filtered under vacuum.

The solution is placed in glass containers or in containers provided with a pump or an adapter to be used as nebulizer solution, colluttory or aerosol solution.

TABLE 2

| | Amount | Concentration % |
|---|---|---|
| Compound | (grams) | (w/w) |
| Flurbiprofen | 0.2500 | 0.25% |
| Glycerol | 10.0000 | 10.0% |
| Ethanol 96° | 9.6000 | 9.6% |
| Liquid sorbitol | 7.0000 | 7.0% |
| Polyoxyethylenated hydrogenated | 2.4000 | 2.4% |
| castor oil 40 | | |
| Sodium saccharin | 0.1500 | 0.15% |
| Methyl parahydroxybenzoate | 0.1000 | 0.10% |
| Propyl parahydroxybenzoate | 0.0200 | 0.02% |
| Tri-rectified peppermint essential | 0.6000 | 0.6% |
| oil | | |
| Patent blue V (E 131) | 0.0006 | 0.006% |
| Anhydrous citric acid | 0.0961 | 0.096% |
| Sodium hydroxide | a.r. at pH 6.5-7.5 | |
| Distilled water | a.r. to 100 ml | |

### Example 3

### Bioavailability of the Liquid Composition Containing FP by Oral Administration

26 persons, healthy volunteers, among whom 16 were males and 10 females, aged between 18 and 55, received two 200 μl nebulizations of the FP solution at 25 mg/ml prepared as in Example 1, three times a day for seven days, and one nebulization on the eighth day.

The plasma concentration was determined for each patient before the first dose and, successively, in the morning of the days 1, 4, 6, 7 and 8 and after 1, 2, 3, 4, 6, 8, 10, 12, 16 and 24 hours since the last treatment.

Each sample was moved into a test tube containing heparin and centrifuged, and the plasma was divided into 500 μl aliquots and freezed at −80° C.

The concentration of FP contained in plasma is represented by the average of the values obtained by the analysis carried out with the method validated in HPLC by using a reversed phase column Hypersil C8 having a size 150×4.6 mm, with 5 μm particles. The FP elution occurs under isocratic conditions with an aqueous solution containing orthophosphoric acid 0.4% (v/v) and acetonitrile in the volumetric ratio, respectively, of 55% and 45%. This method foresees a fluorimetric detection at 260-310 nm and has a detection limit for FP corresponding to 0.025 μg/ml. The following parameters were determined:

Cₘₐₓ: maximal FP plasma concentration observed in plasma.

Tₘₐₓ: time wherein Cₘₐₓ is reached.

AUC: area subtended to the concentration-time curve calculated by means of the linear trapezoidal rule.

The average results of FP plasma concentrations on days 1, 4, 6, 7 are reported in Table 3.

Table 4 reports the average values of Cₘₐₓ, Tₘₐₓ and AUC_{inf.} relating to the eighth day respectively after 1, 2, 3, 4, 6, 8, 10, 12, 16 and 24 hours since the last administration.

TABLE 3

| Day | Time | FP average plasma |
|---|---|---|
| (hours) | (hours) | concentrations (μg/ml) |
| 1 | 0 | 0.000 |
| 4 | 0 | 0.043 |
| 6 | 0 | 0.042 |
| 7 | 0 | 0.039 |
| 8 | 0 | 0.040 |

TABLE 4

| Hours after the last | FP average plasma concentrations |
|---|---|
| administration | (μg/ml) |
| 1 | 0.182 |
| 2 | 0.144 |
| 3 | 0.115 |
| 4 | 0.096 |
| 6 | 0.067 |
| 8 | 0.045 |
| 10 | 0.029 |
| 12 | 0.020 |
| 16 | 0.008 |
| 24 | 0.001 |

### Example 4

### Determination of Local Clinical Tolerability of the Liquid Composition Containing FP

26 persons, healthy volunteers, among whom 16 were males and 10 females, aged between 18 and 55, received two 200 μl nebulizations of the FP solution at 25 mg/ml prepared as in Example 1, three times a day for seven days, and one nebulization on the eighth day.

For the evaluation of local tolerability the researcher had to evaluate the recurrence of erythema, edema, hemorrhagic petechiae and mouth ulcers on days 1, 4, 6, 7 and 8 of the treatment, according to a sequence of none, scarce, moderate and severe.

After each administration of the trial drug, the healthy volunteers were requested to evaluate the sensation of mouth itch, pain or burning possibly felt.

No one of the healthy volunteers showed edema, hemorrhagic petechiae or mouth ulcers. Only a light mouth erythema was observed by a subject on the fourth day and the event healed in two days with no further therapy.

Four subjects reported a total number of three cases of light itch and five cases of light and transitory burning sensation after 572 oral administrations.

### Example 5

### Determination of the Systemic Concentration after Administration of the Liquid Composition Containing FP

The minimum FP concentration was determined by means of a reversed phase chromatographic method, with fluorimetric detector as described in Example 3, and was appreciated only after the fourth day since the beginning of the formulation administration.

Table 5 reports the values obtained after the last administration.

TABLE 5

| Cₘₐₓ | Tₘₐₓ | AUC₀₋₂₄ | AUC_{0-inf.} |
|---|---|---|---|
| (μg/ml) | (hours) | (μg/ml · h) | (μg/ml · h) |
| 0.182 | 1.0 | 0.94 | 1.14 |

The FP half-life (T_{1/2}) was 4.47 and the area subtended to the AUC_{0-inf.} curve turned out to be equal to 1.14 μg/ml·h, ca. 20% larger than the AUC of the last point.

### Example 6

### Determination of Palatability after Administration of the Liquid Composition Containing FP

No one of the healthy volunteers receiving the formulation described in Example 1 complained about the taste or refused to receive the following administrations.

### Example 7

### Determination of Safety of the Liquid Formulation

26 persons, healthy volunteers, among whom 16 were males and 10 females, aged between 18 and 55, received two 200 μl nebulizations of the FP solution at 25 mg/ml described in Example 1, three times a day for seven days, and one nebulization on the eighth day.

No alteration was shown with regard to vital parameters such as blood pressure, heart rate, breathing frequency and body temperature during the objective physical examination and during instrumental evaluations such as electrocardiogram and laboratory analysis.

1. A pharmaceutical composition comprising:
a) a nonsteroidal anti-inflammatory drug;
b) a natural polymer belonging to the xyloglucan family;
c) glycerol and
d) at least one pharmaceutically acceptable excipient
wherein the pharmaceutical composition is an aqueous mucoadhesive.

2. The pharmaceutical composition according to claim 14 comprising flurbiprofen at a concentration between 0.05% and 10.0% by weight, the natural polymer belonging to the xyloglucan family at a concentration between 0.05% and 10.0% by weight and glycerol at a concentration between 5.0% and 70.0% by weight.

3. The pharmaceutical composition according to any one of claim 1 or claim 2, wherein the natural polymer belonging to the xyloglucan family has a viscosity between 150 and 800 mPa sec at a temperature of 25° C. operating with a 200 sec⁻¹ shear rate and a rest time of 15 minutes and an absorbance value at 280 nm lower than 0.5 abs in a 2% by weight aqueous solution at the temperature of 25° C.

4. The pharmaceutical composition according claim 2, wherein the pharmaceutical composition is an oral, topical, vaginal or ophthalmic preparation.

5. A process for producing a pharmaceutical composition comprising a nonsteroidal anti-inflammatory drug wherein the pharmaceutical composition is an aqueous mucoadhesive, said process comprising:
preparing an aqueous solution containing pharmaceutically acceptable salts, adding to said aqueous solution at room temperature and under agitation:
at least one nonsteroidal anti-inflammatory drug, in an amount resulting in a concentration between 0.05% and 10% by weight of said pharmaceutical composition;
a natural polymer belonging to the xyloglucan family in an amount resulting in a concentration between 0.05% and 10.0% by weight of said pharmaceutical composition;
glycerol in an amount resulting in a concentration between 5.0% and 70.0% by weight of said pharmaceutical composition; and
pharmaceutically acceptable excipients.

6. A method of solubilizing nonsteroidal anti-inflammatory drugs, said method comprising combining a nonsteroidal anti-inflammatory drug and a natural polymer belonging to the xyloglucan family in a saline solution; and stirring at room temperature to obtain complete dissolution of said nonsteroidal anti-inflammatory drug.

7. A method of treatment of inflammation or pain in a subject, said method comprising administering the pharmaceutical composition according to any one of claims 1-2 to a subject in need thereof, a therapeutically effective amount of said pharmaceutical composition.

8. The method of treatment according to claim 7, wherein the pharmaceutical composition is administered orally and is characterized to have acceptable palatability.

9. The method of treatment according to claim 7, wherein the pharmaceutical composition is administered ophthalmically.

10. The method of treatment according to claim 7, wherein the pharmaceutical composition is administered vaginally.

11. The method of treatment according to claim 7, wherein the pharmaceutical composition is in the form of spray, mouthwash, emulsion, lavage, aerosol, collutories, ointments, granulates, tablets or plasters.

12. The pharmaceutical composition according to claim 1, further comprising at least one active principal selected from the group consisting of gastroprotectors, antiemetics, antiulcer drugs, anti-stress, ansiolitic agents, antiacids, inhibitors of gastric secretion, antibacterials, mucolytics and antihistamines.

13. The method of treatment according to claim 8, wherein the anti-inflammatory drug is flurbiprofen, and wherein the dosage releases a therapeutically effective amount of flurbiprofen.

14. The pharmaceutical composition according to claim 1, wherein the nonsteroidal anti-inflammatory drug is flurbiprofen or a derivative thereof.

15. The pharmaceutical composition according to any one of claim 1, 2 or 14, wherein the form of the pharmaceutical composition is a spray, mouthwash, emulsion, aerosol or lavage.

16. The pharmaceutical composition according to claim 8, wherein the oral preparation comprises sweetener agents and/or natural fragrance for oral use.

17. The method of treatment according to claim 7, wherein the nonsteroidal anti-inflammatory drug is flurbiprofen or derivatives thereof.
